(19) United States
(12) Patent Application Publication
(10) Pub. No.: US 2008/0214857 A1
(43) Pub. Date: Sep. 4, 2008

(54) Method Of Reducing A Functional Group In An Oxidized Form

(75) Inventors: Gerard Mignani, Lyon (FR); Mikael Berthod, Lyon (FR); Marc Lemaire, Villeurbanne (FR)

Correspondence Address: BUCHANAN, INGERSOLL & ROONEY PC, POST OFFICE BOX 1404, ALEXANDRIA, VA, 22313-1404, US

(73) Assignees: RHODIA CHIMIE, Aubervilliers (FR); CENTRE NATIONAL DE LA RECHERCHE SCIENTIFIQUE, Paris, Cedex (FR)

(21) Appl. No.: 11/587,397

(22) PCT Filed: Apr. 20, 2005
(86) PCT No.: PCT/FR2005/000975

§ 371 Date: Sep. 10, 2007

(30) Foreign Application Priority Data
Apr. 23, 2004 (FR) 0404326

(51) Int. Cl. C07C 255/00; C07C 67/00; C07F 9/28; C07C 325/02; C07C 233/00

(52) U.S. Cl. 558/303; 560/1; 564/123; 568/14; 568/20

(57) ABSTRACT

A novel method of reducing a functional group in an oxidised form. The invention relates more particularly to the reduction of aldehyde, ketone, ester, lactone, nitrile or phosphine oxide groups. The reduction method according to the invention is characterised in that it comprises exposing the substrate including the functional group to be reduced to the presence of a siloxane-type compound of the following formula (I), combined with a Lewis acid-type catalyst. In said formula (I):—R1 and R2, which are the same or different, are an alkyl, cycloalkyle or aryl group, —X is a digit from 0 to 50.

The present invention relates to a novel method of reducing a functional group in an oxidized form.

The invention applies more particularly to the reduction of aldehyde, ketone, ester, lactone, nitrile or phosphine oxide groups.

The reduction of a functional group is a very important reaction in the field of organic chemistry.

Thus, the reduction of phosphine oxides to phosphine has been largely described.

Lithium aluminum hydride is often recommended, and in particular by Kawakami, Y. et al (Synt. Commun, 1983, 13, 427-434). However, this reactant is not easy to handle because it is dangerous.

A new triethoxysilane/titanium (IV) isopropoxide system has been described by Buchwald (J.A.C.S. 1991, 113, 5093), but triethoxysilane is not an ideal reactant because it is very toxic and dangerous.

Coumbe, T. et al (Tetrahedron Letters 1994, 35, 625-628) have described an alternative which consists in using a polymethylhydrosiloxane (PMHS). Admittedly, the latter is a relatively inexpensive, non-volatile and less toxic reactant, but it requires the use of a large excess of titanium (IV) isopropoxide (100 mol %), and use on an industrial scale is difficult due to the formation of a gel.

As regards the reduction of carbonyl functional groups, such as aldehydes, ketones, esters or lactones, it is known practice, according to WO 96/12694, to use a silane derivative and a metal hydride, the latter being obtained in situ or ex situ from a metal complex or salt by reaction with a reducing agent.

Trialkylsilanes, dialkylsilanes, trialkoxysilanes and polymethylhydrosiloxane (PMHS) are recommended as silane agents.

Another drawback of the method described is that it uses reducing agents of the hydride type: lithium hydride, sodium hydride, potassium hydride, boron hydride, metal loborohydride, aluminum hydride or organomagnesium or organolithium compounds which cannot be readily handled since they are highly reactive and dangerous.

The objective of the present invention is to provide a method which overcomes the abovementioned drawbacks.

A method has now been found, and it is this which forms the subject of the present invention, which is a method of reducing an oxidized functional group, present in a substrate, to a lower oxidation state, characterized in that it comprises exposing the substrate to a siloxane-type compound corresponding to the following formula (I), combined with an effective amount of a Lewis acid-type catalyst

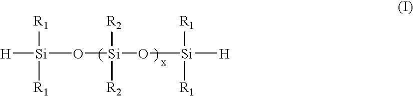

in which said formula:
R₁ and R₂, which may be identical or different, represent an alkyl, cycloalkyl or aryl group,
x is a number ranging from 0 to 50.

In the context of the invention, the term “alkyl” is intended to mean a linear or branched hydrocarbon-based chain having from 1 to 10 carbon atoms, and preferably from 1 to 4 carbon atoms.

Examples of preferred alkyl groups are, in particular, methyl, ethyl, propyl, isopropyl, butyl, isobutyl and t-butyl.

The term “cycloalkyl” is intended to mean a monocyclic, cyclic hydrocarbon-based group comprising 5 or 6 carbon atoms, preferably a cyclopentyl or cyclohexyl group.

The term “aryl” is intended to mean an aromatic monocyclic or polycyclic group, preferably monocyclic or bicyclic, comprising from 6 to 12 carbon atoms, preferably phenyl.

The siloxane-type compounds which are used in the method of the invention correspond to formula (I) in which R₁ and R₂ are identical and represent more particularly an alkyl group having from 1 to 4 carbon atoms.

R₁ and R₂ preferably represent a methyl group.

As regards x, it is preferably between 0 and 10, and even more preferably equal to 0 or 1.

Among the compounds of formula (I), that which is preferred is the following compound:

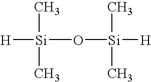

referred to by the abbreviation TMDS, tetramethyl-disiloxane.

In accordance with the method of the invention, the reduction of various functional groups is carried out, and most particularly the following groups:
functional groups comprising a carbonyl group, such as: aldehyde, ketone, carboxylic acid, ester or amide;
functional groups comprising a nitrogen atom, such as nitrile, imine, nitro or nitrogen oxide;
functional groups comprising a sulfur atom, such as sulfoxide or sulfone;
functional groups comprising a phosphorus atom, such as a phosphine oxide or phosphine sulfide.

The various abovementioned groups can be carried by an aliphatic chain or a ring, but it is also possible for them to be included in a ring, such as, for example, a cyclic ketone, a lactone or a lactam.

Thus, in a symbolic manner, the various substrates comprising the functions capable of being reduced can be represented in this way:

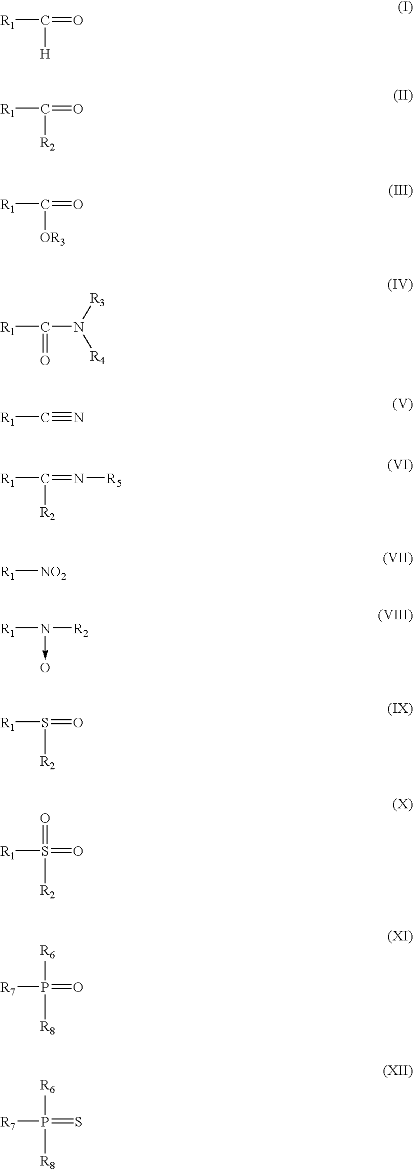

in which said formulae,
R₁ to R₈ represent a hydrocarbon-based group having from 1 to 20 carbon atoms,
R₃, R₄ and R₅ also represent a hydrogen atom,
at most one of the groups R₆, R₇ and R₈ represents a hydrogen atom,
R₁ and R₂, R₁ and R₅, R₆ and R₇, R₇ and R₈, and R₆ and R₈ can be linked together so as to form a ring.

It should be noted that the substrate can be mono-functional or polyfunctional (most commonly bifunctional).

Thus, the same function can be present several times (for example, diketone, diphosphine in dioxide or disulfide form) or functions of different nature can be present (for example, nitrile function and phosphine oxide).

In said formulae (I) to (XII), R₁ and R₂ represent a hydrocarbon-based group of any nature. Preferred meanings will be specified in the text, but without them being in any way limiting in nature.

More specifically, R₁ and R₂ represent a hydrocarbon-based group having from 1 to 20 carbon atoms, which can be a saturated or unsaturated, linear or branched, acyclic aliphatic group; a saturated, unsaturated or aromatic, monocyclic or polycyclic, carbocyclic or heterocyclic group; a saturated or unsaturated, linear or branched aliphatic group carrying a cyclic substituent.

R₁ and R₂ preferably represent a saturated, linear or branched, acyclic aliphatic group preferably having from 1 to 12 carbon atoms.

The invention does not exclude the presence of another unsaturation on the hydrocarbon-based chain, such as one or more double bonds, which may or may not be conjugated, or alternatively a triple bond.

The hydrocarbon-based chain can be optionally interrupted with a heteroatom (for example, oxygen or sulfur) or with a functional group insofar as the latter does not react, and mention may in particular be made of a group such as, in particular, ether or alcohol.

The hydrocarbon-based chain can optionally carry one or more substituents insofar as they do not react under the reaction conditions, and mention may in particular be made of a halogen atom or a trifluoromethyl group.

The saturated or unsaturated, linear or branched, acyclic aliphatic group can optionally carry a cyclic substituent. The term “ring” is intended to mean a saturated, unsaturated or aromatic, carbocyclic or heterocyclic ring.

The acyclic aliphatic group may be connected to the ring via a valency bond, a heteroatom or a functional group, such as oxy, carbonyl, carboxyl, sulfonyl, etc.

As examples of cyclic substituents, cycloaliphatic, aromatic or heterocyclic substituents may be envisioned, in particular cycloaliphatic substituents comprising 6 carbon atoms in the ring or benzene substituents, these cyclic substituents themselves optionally carrying any substituent insofar as they do not hinder the reactions involved in the method of the invention. Mention may in particular be made of alkyl or alkoxy groups having from 1 to 4 carbon atoms.

Among the linear or branched aliphatic groups, alkyl groups having from 1 to 10 carbon atoms are in particular targeted.

Among the aliphatic groups carrying a cyclic substituent, aralkyl groups having from 7 to 12 carbon atoms, in particular benzyl or phenylethyl, are more particularly envisioned.

In the formulae, R₁ and R₂ may represent a monocyclic carbocyclic group. The number of carbon atoms in the ring can vary to a large extent, from 3 to 8 carbon atoms, but it is preferably equal to 5 or 6 carbon atoms.

The carbocycle can be saturated or can comprise 1 or 2 unsaturations in the ring, preferably 1 or 2 double bonds.

As preferred examples of groups R₁ and R₂, mention may be made of cyclohexyl or cyclohexenyl groups.

R₁ and R₂ can also represent, independently of one another, a polycyclic hydrocarbon-based group consisting of at least 2 saturated and/or unsaturated carbocycles or at least 2 carbocycles of which only one is aromatic and which form with one another orthocondensed or ortho- and pericondensed systems. Generally, the rings are C₃ to C₈, preferably C₆, rings. As more specific examples, mention may be made of the bornyl group or the tetrahydronaphthalene group.

R₁ and R₂ may represent an aromatic carbocyclic group having from 4 to 8 carbon atoms, preferably a phenyl group.

R₁ and R₂ may also represent a polycyclic aromatic carbocyclic group; it being possible for the rings to form with one another orthocondensed or ortho- and pericondensed systems. Mention may more particularly be made of a naphthalene group.

When R₁ and R₂ represent a saturated or unsaturated, monocyclic carbocyclic group, it is possible for one or more of the carbon atoms of the ring to be replaced with a heteroatom, preferably oxygen, nitrogen or sulfur, or with a functional group, preferably carbonyl or ester, thus producing a monocyclic heterocyclic compound. The number of atoms in the ring can vary to a large extent, from 3 to 8, but it is preferably equal to 5 or 6 atoms.

R₁ and R₂ can also represent a polycyclic aromatic heterocyclic group defined as being either a group consisting of at least 2 aromatic or nonaromatic heterocycles containing at least one heteroatom in each ring and forming with one anther orthocondensed or ortho- and pericondensed systems, or a group consisting of at least one aromatic or nonaromatic hydrocarbon-based ring and at least one aromatic or nonaromatic heterocycle forming with one another orthocondensed or ortho- and pericondensed systems.

By way of examples of groups R₁ and R₂ of heterocyclic type, mention may be made, inter alia, of furyl, thienyl, isoaxazolyl, furazanyl, isothiazolyl, pyridyl, pyridazinyl, pyrimidinyl and pyranyl groups and quinolyl, naphthyridinyl, benzopyranyl and benzofuranyl groups.

It should be noted that, if the groups R₁ and R₂ comprise any ring, it is possible for this ring to carry a substituent. The substituent can be of any nature, insofar as it does not interfere in terms of the desired product. The substituents most commonly carried by the ring are one or more alkyl or alkoxy groups preferably having from 1 to 4 carbon atoms, preferably methyl or methoxy, an alkenyl group, preferably an isopropenyl group, a halogen atom, preferably chlorine or bromine, a trihalomethyl group, preferably trifluoromethyl, and functional groups, more particularly nitrile or ester (preferably of C₁-C₄ lower alkyl).

Among all the abovementioned groups R₁ and R₂, R₁ and R₂ preferably represent a phenyl group optionally carrying an alkyl or alkoxy group having from 1 to 4 carbon atoms, or a trifluoromethyl group.

R₁ and R₂ can be linked via a saturated or unsaturated aliphatic chain so as to constitute a saturated or unsaturated carbocycle or heterocycle having from 3 to 20 atoms, which is monocyclic or polycyclic comprising two or three rings: it being possible for the adjacent rings to be aromatic in nature. In the case of polycyclic compounds, the number of atoms in each ring preferably ranges between 3 and 6.

R₁ and R₂ can form an alkylene or alkenylene chain having from 4 to 6 carbon atoms, and preferably 5 carbon atoms.

In this case, a cyclic ketone corresponding to formula (II), a lactone for formula (III) and a lactam for formula (IV) are obtained.

It is also possible, for formulae (IX) and (X), for the sulfoxide and sulfone group to also be included in a ring.

As regards the groups R₃, R₄ and R₅, they represent a hydrogen atom or an alkyl, alkenyl, aryl or arylalkyl group.

As regards R₆, R₇ and R₈, they are more particularly an alkyl, alkenyl, aryl or arylalkyl group.

Examples of substrates which can be reduced are given hereinafter.

As examples of substrates of aldehyde type and illustrated by formula (I), mention may be made of saturated aldehydes such as butanal, pentanal, hexanal, heptanal, octanal, decanal or dodecanal; unsaturated aldehydes such as acrolein, methacrolein, crotonaldehyde, prenal, citral, retinal, campholenic aldehyde, cinnamic aldehyde, hexylcinnamic aldehyde, formylpinane and nopal; aromatic aldehydes such as benzaldehyde, salicylic aldehyde, vanillin or veratraldehyde.

As examples of ketones corresponding to formula (II), mention may in particular be made of hexan-2-one, octan-2-one, nonan-4-one, dodecan-2-one, methyl vinyl ketone, mesityl oxide, acetophenone, cyclopentanone, cyclohexanone, cyclododecanone, cyclohex-1-en-3-one, isophorone, oxyphorone, carvone and camphor.

As examples of substrates illustrating formula (III), mention may be made of acids and their ester derivatives (alkyl, preferably methyl, or aryl) of the following acids: acetic acid, propionic acid, butyric acid, isobutyric acid, lactic acid, tartaric acid, benzoic acid, salicylic acid, p-hydroxybenzoic acid, vanillic acid, veratric acid, acrylic acid, methacrylic acid, crotonic acid, hexenoic acid, fumaric acid, citraconic acid and cinnamic acid. Mention may also be made of saturated fatty acids such as lauric acid, myristic acid, palmitic acid, stearic acid or sebacic acid; unsaturated fatty acids, and more particularly unsaturated fatty acids having a sole double bond, such as linderic acid, myristoleic acid, palmitoleic acid, oleic acid, petroselenic acid, doeglic acid, gadoleic acid, erucic acid; unsaturated fatty acids having two double bonds, such as linoleic acid; unsaturated fatty acids having 3 double bonds, such as linolenic acid; unsaturated fatty acids having more than 4 double bonds, such as isanic acid, stearodonic acid, arachidonic acid, and chypanodonic acid; unsaturated fatty acids carrying a hydroxyl group, such as ricinoleic acid, and mixtures thereof.

As regards the amides of formula (IV), mention may in particular be made of N-alkylcarboxamides or N-benzyl-carboxamides, such as, in particular, N-ethylacetamide or N-benzylacetamide.

The lactones used as starting substrates are more particularly lactones having from 3 to 12 carbon atoms in the ring, preferably γγ-valerolactone or 4-methyl-butyrolactone, δδ-valerolactone or 2-methylbutyrolactone, εδ-valerolactone, ωδ-valerolactone, 3-ethylpropiolactone, 2-ethylpropiolactone, 2,3-dimethyllactone, caprolactone or 12-dodecanelactone.

As lactams, mention may be made of lactams having from 3 to 12 atoms in the ring, and more particularly caprolactam, δδ-valerolactam, εδ-valerolactam, γδ-valerolactam, ωδ-valerolactam, 11-undecalactam and 12-dodecanelactam.

As examples of substrates corresponding to formula (V), mention may in particular be made of aliphatic or aromatic nitriles, preferably acetonitrile, propionitrile, butanenitrile, isobutanenitrile, pentanenitrile, 2-methyl-glutaronitrile, adiponitrile, benzonitrile, tolunitrile, malonitrile or 1,4-benzonitrile.

The method of the invention is entirely suitable for carrying out the reduction of phosphine oxides or of diphosphine oxides, regardless of whether the phosphines are chiral or nonchiral.

Examples of phosphine oxides which can be reduced according to the invention are given hereinafter, which examples are not limiting insofar as the method of the invention applies to any substrate comprising the group

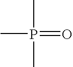

As phosphine oxides which can be reduced, mention may in particular be made of those of formula:

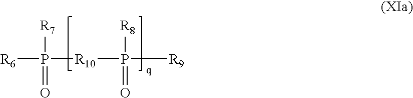

in which said formula:
q is equal to 0 or 1;
the groups R₆, R₇, R₈ and R₉, which may be identical or different, represent:
   an alkyl group having from 1 to 12 carbon atoms,
   a cycloalkyl group having 5 or 6 carbon atoms,
   a cycloalkyl group having 5 or 6 carbon atoms, substituted with one or more alkyl groups having 1 to 4 carbon atoms, or alkoxy groups having from 1 to 4 carbon atoms,
   a phenylalkyl group in which the aliphatic portion contains from 1 to 6 carbon atoms,
   a phenyl group,
   a phenyl group substituted with one or more alkyl groups having from 1 to 4 carbon atoms or alkoxy groups having from 1 to 4 carbon atoms, one or more halogen atoms or a trifluoromethyl group, or with a solubilizing group;
the group R₁₀ represents:
   a valency bond or a saturated or unsaturated, linear or branched, divalent hydrocarbon-based group having from 1 to 6 carbon atoms,
   an aromatic group of formula:

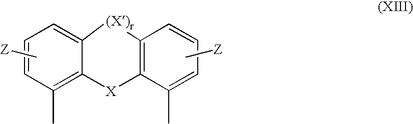

in which:
   Z represents an alkyl group having from 1 to 10 carbon atoms, a halogen atom or a trifluoromethyl group,
   X represents an oxygen or sulfur atom or a linear or branched alkylene group having from 1 to 3 carbon atoms,
   if r is equal to 1, X′ represents a valency bond, an oxygen, sulfur or silicon atom or a linear or branched alkylene group having from 1 to 3 carbon atoms,
   if r is equal to 0, the two rings are not linked;
carbon atoms;

It is also possible for at least one of the three hydrocarbon-based groups connected to the phosphorous to carry a solubilizing group S, which may be one or more hydroxyl groups and/or functional groups of anionic type, in particular SO₂W, SO₃W or COOW in which W represents a hydrogen atom or an alkali metal, preferably sodium, a phosphonate group or an ammonium N⁺R₃ or phosphonium P⁺R₃ group in which the groups R most commonly represent an alkyl group having from 1 to 4 carbon atoms or a benzyl group.

By way of examples of phosphine oxides, mention may be made, in a nonlimiting manner, of: phosphine oxides derived from the following phosphines: tricyclohexyl-phosphine, trimethylphosphine, triethylphosphine, tri-n-butylphosphine, triisobutylphosphine, tri-tert-butyl-phosphine, tribenzylphosphine, dicyclohexylphenyl-phosphine, triphenylphosphine, dimethylphenylphosphine, diethylphenylphosphine, di-tert-butylphenylphosphine, tri(p-tolyl)phosphine, isopropyldiphenylphosphine, tris-(pentafluorophenyl)phosphine, tri(o-tolyl)phosphine, bisdiphenylphosphinomethane, bisdiphenylphosphinoethane, bisdiphenylphosphinopropane, bisdiphenylphosphinobutane, bisdiphenylphosphinopentane, bis[(2-diphenyl-phosphino)phenyl]ether (DPEPHOS), 4,5-bis(diphenyl-phosphino)-9,9-dimethylxanthene (XANTPHOS), and sodium triphenylphosphinotrimetasulfonate (TPPTS).

Phosphine oxides of another type which can be reduced are those which correspond to formula (XIb) and which are partly described in WO-A 00/52081:

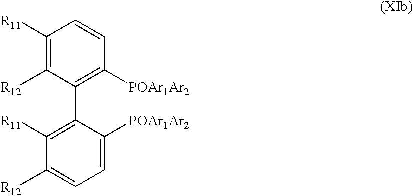

in which:
Ar₁ and Ar₂ independently represent a saturated or unsaturated, linear or branched, aliphatic hydrocarbon-based group; a saturated, unsaturated or aromatic carbocycle;
R₁₁ and R₁₂ independently represent a hydrogen atom; a group Z; or a group —XZ, where X represents O, S or —NT; and

Z and T are independently chosen from a saturated aliphatic hydrocarbon-based group optionally interrupted with O, S and/or N; a saturated, unsaturated or aromatic carbocyclic group; or a saturated aliphatic hydrocarbon-based group substituted with one or more saturated, unsaturated or aromatic carbocyclic groups, in which the aliphatic group is optionally interrupted with O, S and/or N; it being understood that T can also represent a hydrogen atom; or else
two groups R₁₁ and R₁₂, attached to the same phenyl ring, together form an unsaturated or aromatic carbocycle, or else together form an unsaturated or aromatic heterocycle.

In the context of the invention, the expression “saturated or unsaturated, linear or branched, aliphatic hydrocarbon-based group” is intended to mean an optionally substituted, saturated or unsaturated, linear or branched C₁-C₂₅, preferably C₁-C₁₂ group, and the term “carbocyclic group” is intended to mean an optionally substituted, monocyclic or polycyclic, preferably C₃-C₅₀, and more preferably C₃-C₁₈, group.

When the carbocyclic group comprises more than one cyclic ring (in the case of polycyclic carbocycles), the cyclic rings can be condensed (o-condensed or pericondensed) in pairs or attached in pairs via σ bonds.

The carbocyclic group can comprise a saturated portion and/or an aromatic portion and/or an unsaturated portion.

Examples of saturated carbocyclic groups are cycloalkyl groups.

Preferably, the cycloalkyl groups are C₃-C₁₈ groups, better still C₃-C₁₀ groups. Mention may in particular be made of cyclopentyl, cyclohexyl, cycloheptyl, cyclooctyl, adamantyl or norbornyl groups.

The unsaturated carbocycle, or any unsaturated portion, has one or more ethylenic unsaturations. It preferably has from 6 to 50 carbon atoms, better still from 6 to 20, for example from 6 to 18.

Examples of unsaturated carbocycles are C₆-C₁₀ cycloalkenyl groups.

Examples of aromatic carbocyclic groups are (C₆-C₁₈)aryl groups, and in particular phenyl, naphthyl, anthryl and phenanthryl.

The term “aliphatic hydrocarbon-based group” is intended to mean an optionally substituted, saturated, linear or branched C₁-C₂₅, preferably C₁-C₁₂, and even more preferably C₁-C₆, group.

The substituents of the carbocyclic groups (St1) can be saturated aliphatic hydrocarbon-based groups optionally interrupted with O, S and/or N, or groups —XZ in which X and Z are as defined above.

The substituents of the hydrocarbon-based aliphatic groups (St2) are saturated or unsaturated carbocyclic groups which are themselves optionally substituted with one or more of the substituents (St1) defined above.

Preferably, Ar₁ and Ar₂ independently represent (C₃-C₈)cycloalkyl or (C₆-C₁₈)aryl, optionally substituted with one or more (C₁-C₆)alkyl and/or (C₁-C₆)alkoxy; and R₁₁ and R₁₂ independently represent a hydrogen atom; (C₃-C₈)cycloalkyl; (C₁-C₆)alkyl; (C₁-C₆)alkoxy or (C₆-C₁₈)aryl, the cycloalkyl and aryl groups being optionally substituted with (C₁-C₆)alkyl and/or (C₁-C₆)alkoxy.

When R₁₁ and R₁₂ together form an unsaturated carbocycle or heterocycle, the latter preferably has a single unsaturation which is that shared with the phenyl ring carrying the groups R₁₁, and R₁₂.

The aromatic carbocycles that R₁₁and R₁₂ together form are preferably as defined above.

The unsaturated carbocycles that R₁₁ and R₁₂ together form are monocyclic or polycyclic, the definition of these terms being as proposed above. These carbocycles preferably comprise from 6 to 50 carbon atoms, better still from 6 to 20 carbon atoms. Examples thereof are in particular C₆-C₁₀ cycloalkenyl.

According to the invention, the term “heterocycle” is intended to mean monocyclic or polycyclic groups, and in particular monocyclic, bicyclic or tricyclic groups, comprising one or more heteroatoms chosen from O, S and/or N, preferably 1 to 4 heteroatoms.

When the heterocycle is polycyclic, the latter can consist of several monocycles condensed in pairs (orthocondensed or pericondensed) and/or of several monocycles attached in pairs via σ bonds.

Preferably, the monocycles or the monocycle constituting the heterocycle have from 5 to 12 ring members, better still from 5 to 10 ring members, for example 5 or 6 ring members.

When R₁₁ and R₁₂ form a heterocycle, the latter comprises an unsaturated portion and/or an aromatic portion, it being understood that the unsaturated portion preferably comprises a sole double bond.

Heterocycles which are particularly preferred are especially pyridine, furan, thiophene, pyrrole, benzofuran and benzothiophene.

In the context of the invention, monocyclic or bicyclic carbocycles and heterocycles are preferred.

According to the invention, when R₁₁ and R₁₂ together form a carbocycle or heterocycle, the latter can optionally be substituted with one or more substituents (St1) as defined above.

It should be noted that the invention does not exclude the possibility of the two benzene rings present in formula (XIb) carrying other substituents. As examples, reference may be made to the definition of X₁ and X₂ given for formula (XIc).

The phosphine oxides used preferably correspond to formula (XIb) in which:
Ar₁ and Ar₂ independently represent a (C₁-C₆)alkyl group, preferably a t-butyl group; a saturated, unsaturated or aromatic monocyclic carbocycle optionally substituted with one or more (C₁-C₆)alkyl or (C₁-C₆)alkoxy groups and having from 3 to 8 carbon atoms;
R₁₁ and R₁₂ are independently chosen from a hydrogen atom, a (C₁-C₆)alkyl group or a (C₁-C₆)alkoxy group; or else
R₁₁ and R₁₂ form, together with the carbon atoms which carry them, (i) an unsaturated or aromatic, monocyclic or polycyclic carbocycle having from 5 to 13 carbon atoms, or (ii) an unsaturated or aromatic, monocyclic or polycyclic heterocycle having from 4 to 12 carbon atoms and one or more heteroatoms chosen from O, S and N, said heterocycle and carbocycle being optionally substituted with one or more (C₁-C₆)alkyl or (C₁-C₆)alkoxy.

Even more preferably, Ar₁ and Ar₂ are identical and represent a phenyl group optionally substituted with one or more (C₁-C₆)alkyl or (C₁-C₆)alkoxy; or a (C₄-C₈)cycloalkyl group optionally substituted with one or more (C₁-C₆)alkyl groups.

Ar₁ and Ar₂ are identical and represent cyclohexyl, phenyl or tolyl.

R₁₁ and R₁₂ are independently chosen from a hydrogen atom, (C₁-C₆)alkyl or (C₁-C₆)alkoxy, or else R₁₁ and R₁₂ form, together with the carbon atoms which carry them, a cyclohexenyl, with a sole unsaturation, optionally substituted with one or more (C₁-C₆)alkyl or (C₁-C₆)alkoxy; or phenyl optionally substituted with one or more (C₁-C₆)alkyl or (C₁-C₆)alkoxy.

As examples of phosphine oxides which can be reduced, a first group of preferred compounds consists of the compounds of formula (XIb) for which R₁₁ and R₁₂ are a hydrogen atom, a (C₁-C₆)alkyl group (preferably methyl) or a (C₁-C₆)alkoxy group (preferably methoxy).

Mention may in particular be made of:

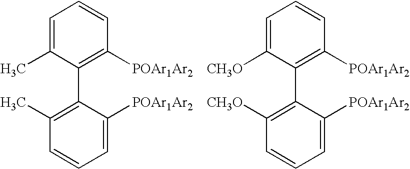

A second group of preferred compounds consists of the compounds of formula (XIb) for which R₁₁ and R₁₂ together form optionally substituted (C₃-C₁₁)cycloalkenyl, optionally substituted (C₆-C₁₀)aryl or (C₄-C₈)heteroaryl comprising 1 or 2 endocyclic heteroatoms, said heteroaryl being optionally substituted.

R₁₁ and R₁₂ together represent an optionally substituted phenyl group or a cycloalkenyl group.

Thus, particularly concerned are diphosphine oxides in which the naphthyl groups are substituted in the 4,4′- or 5,5′- or 6,6′-position with atoms or functional groups and which can be represented by the following formula:

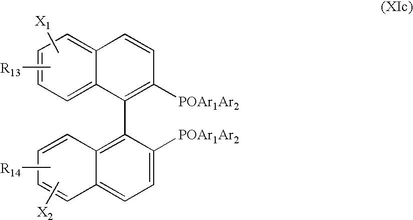

R₁₃ and R₁₄, which may be identical or different, represent a hydrogen atom or a substituent,
Ar₁ and Ar₂ independently represent an alkyl, alkenyl, cycloalkyl, aryl or arylalkyl group,
X₁ and X₂, which may be identical or different, represent:
   a group R, alkyl, alkenyl, alkynyl, cycloalkyl, aryl or arylalkyl,
   an alkyl group substituted with one or more halogen atoms, preferably fluorine, or with nitro or amino groups,
   a halogen atom chosen from bromine, chlorine or iodine,
   an —OH group,
   an —O—Rₐ group,
   an —O—CORₐ group,
   an —S—Rₐ group,
   an —SO₃M group,
   a —CN group,
   a group derived from the nitrile group such as:
      a —CH₂—NH₂ group,
      a —COOH group,
   a group derived from the carboxylic group, such as:
      a —COORₐ group,
      a —CH₂OH group,
      a —CO—NH—R_{b} group,
   a group derived from the aminomethyl group, such as:
      a —CH₂—NH—CO—R_{b} group,
      a —CH₂—NH—CO—NH—R_{b} group,
      a —CH₂—N═CH—Rₐ group,
      a —CH₂—NH₄⁺ group,
   a group comprising a nitrogen atom, such as:
      an NH₂ group,
      an —NHRₐ group,
      an —N(Rₐ)₂ group,
      an —N═CH—Rₐ group,
      an —NH—NH₂ group,
      an —N≡N⁺≡N⁻ group,
      a magnesium or lithium atom;
   in the various formulae, Rₐ represents an alkyl, cycloalkyl, arylalkyl or phenyl group and R_{b} has the meaning given for Rₐ and also represents a naphthyl group and M represents a metal cation, preferably alkali metal cation, in particular sodium cation.

In formula (XIc), the term “alkyl” is intended to mean a C₁-C₁₅, preferably C₁-C₁₀, linear or branched hydrocarbon-based chain. Examples of preferred alkyl groups are in particular methyl, ethyl, propyl, isopropyl, butyl, isobutyl and t-butyl.

The term “alkenyl” is intended to mean a C₂-C₁₅ linear or branched hydrocarbon-based group comprising one or more double bonds, preferably one or two double bonds.

The term “alkynyl” is intended to mean a C₂-C₁₅ linear or branched hydrocarbon-based group comprising one or more triple bonds, preferably one triple bond.

The term “cycloalkyl” is intended to mean a C₃-C₈ monocyclic cyclic hydrocarbon-based group, preferably a cyclopentyl or cyclohexyl group, or a C₄-C₁₈ polycyclic (bicyclic or tricyclic) cyclic hydrocarbon-based group, in particular adamantyl or norbornyl.

The term “aryl” is intended to mean an aromatic, monocyclic or polycyclic, preferably monocyclic or bicyclic, C₆-C₂₀ group, preferably phenyl or naphthyl. When the group is polycyclic, i.e. it comprises more than one cyclic ring, the cyclic rings can be condensed in pairs or attached in pairs via σ bonds. Examples of (C₆-C₁₈)aryl groups are in particular phenyl, naphthyl, anthryl and phenanthryl.

The term “arylalkyl” is intended to mean a linear or branched hydrocarbon-based group carrying an aromatic monocyclic C₇-C₁₂ ring, preferably benzyl.

The various groups X₁ and X₂ are advantageously in the 6,6′-, 5,5′- or 4,4′-position.

As examples of substrates, mention may be made of the oxides corresponding to diphosphines substituted in the 6-position and 6′-position and which are described in patents WO-A 00/49028 and WO-A 01/74828.

For the diphosphine oxides substituted in the 5-position and 5′-position, reference may be made to those corresponding to the diphosphines described in applications FR-03/04392 and FR-02/16086.

For the diphosphine oxides substituted in the 4-position and 4′-position, reference may be made to those corresponding to the diphosphines described in applications FR-03/04391 and FR-02/16087.

The method applies most particularly to diphosphine oxides which have one or two nitrile groups, because there is at the same time a reduction of the nitrile groups and of the diphosphine oxide function.

Examples of said diphosphine oxides are given.

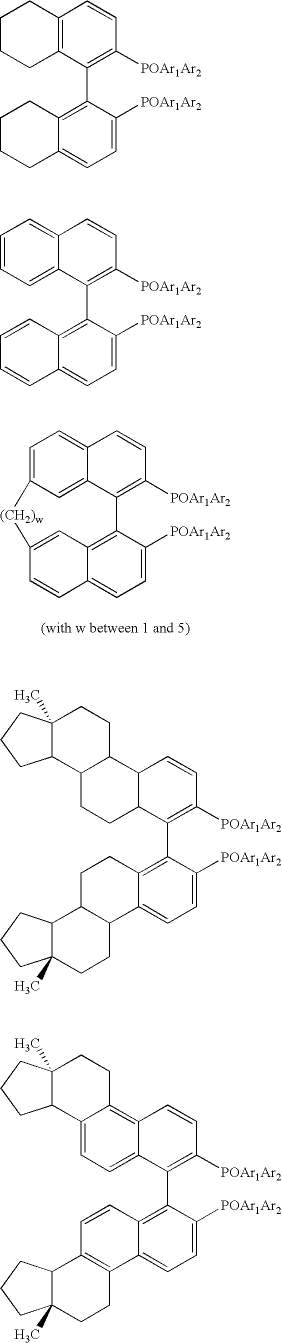

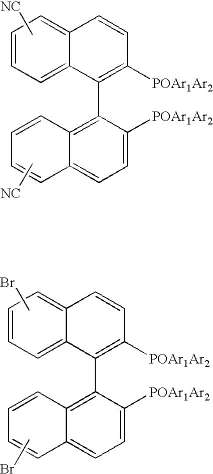

The invention applies particularly to BINAP, i.e. 2,2′-bis(diphenylphosphino)-1,1′-binaphthyl, and to derivatives thereof substituted in the 6- and 6′-position, 5- and 5′-position or 4- and 4′-position.

The method of the invention is also suitable for diphosphine oxides corresponding to the following formula:

R₁₅R₁₆OP-A-POR₁₅R₁₆ (XId)

in which:
A represents a saturated divalent aliphatic hydrocarbon-based group; a saturated or aromatic divalent carbocyclic group; a saturated divalent aliphatic hydrocarbon-based group interrupted with a saturated or aromatic divalent carbocyclic group;
R₁₅ and R₁₆ are different and represent a saturated aliphatic hydrocarbon-based group; an aromatic carbocyclic or aromatic heterocyclic group.

The saturated aliphatic hydrocarbon-based groups and the aromatic carbocyclic and heterocyclic groups representing R₁₅ and R₁₆ are as defined above. They can be substituted with one or more substituents -Z or —XZ in which X and Z are as defined above.

In formula (XId), the various symbols represent more particularly:
A represents a C₁-C₆ alkylene chain optionally substituted with one or more (C₁-C₆)alkoxy, di(C₁-C₆)alkylamino or (C₁-C₆)alkylthio groups; a (C₃-C₈)cycloalkylene group optionally substituted with one or more (C₁-C₆)alkoxy, di(C₁-C₆)alkylamino or (C₁-C₆)alkylthio groups; a (C₆-C₁₀)arylene group optionally substituted with one or more (C₁-C₆)alkoxy, di(C₁-C₆)alkylamino or (C₁-C₆)alkylthio groups; or a group —(CH₂)ⱼ—B″-(CH₂)ⱼ— where j represents an integer from 1 to 3 and B″ represents (C₃-C₈) cycloalkylene optionally substituted with one or more (C₁-C₆)alkoxy, di(C₁-C₆)alkylamino or (C₁-C₆)alkylthio, or (C₆-C₁₀)arylene optionally substituted with one or more (C₁-C₆) alkoxy, di(C₁-C₆)alkylamino or (C₁-C₆)alkylthio;
R₁₅ and R₁₆ are different and represent an aromatic monocyclic heterocycle having from 3 to 7 carbon atoms and one or more heteroatoms chosen from O, N and S; a (C₆-C₁₀)aryl group, said heterocycle and said aryl group being optionally substituted with one or more (C₁-C₆)alkyl or (C₁-C₆)alkoxy groups; or else (C₁-C₆)alkyl optionally substituted with one or more (C₁-C₆)alkoxy.

Even more preferably, A represents ethylene and R₁₅ and R₁₆ are independently chosen from phenyl optionally substituted with one or more (C₁-C₆)alkyl or (C₁-C₆)alkoxy.

A more specific example is given below:

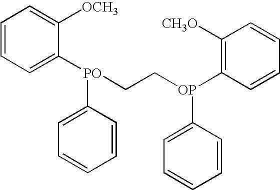

Substrates of another type which can be reduced are those which correspond to the following formula:

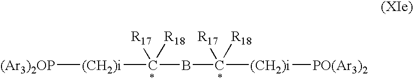

in which:
* denotes an asymmetrical center;
i represents 0 or 1;
R₁₇ and R₁₈ independently represent a hydrogen atom or a saturated aliphatic hydrocarbon-based group, or else the groups R₁₈ are as defined above and the groups R₁₇ together form a saturated divalent aliphatic hydrocarbon-based chain optionally interrupted with two groups X, X being as defined above for formula (XIb), preferably with two identical groups X;
B represents a bond or else is as defined above for A in formula (XId);
Ar₃ is as defined above for R₁₅ and R₁₆.

In formula (XIe), the various symbols represent more particularly:
R₁₇ and R₁₈ are independently chosen from a hydrogen atom and a (C₁-C₆)alkyl group; or else the two groups R₁₇ together form a (C₁-C₆)alkylene chain optionally interrupted with two oxygen or sulfur atoms, and R₁₈ is as defined above;
B represents a bond or else is as defined above for A;
Ar₃ represents an aromatic monocyclic heterocycle having from 3 to 7 carbon atoms and one or more heteroatoms chosen from O, N and S; or a (C₆-C₁₀)aryl group, said heterocycle and said aryl group being optionally substituted with one or more (C₁-C₆)alkyl or (C₁-C₆)alkoxy groups; or else (C₁-C₆)alkyl optionally substituted with one or more (C₁-C₆)alkoxy.

Even more preferably, Ar₃ represents a phenyl group optionally substituted with one or more (C₁-C₆)alkyl or (C₁-C₆)alkoxy.

Examples of said phosphine oxides are given.

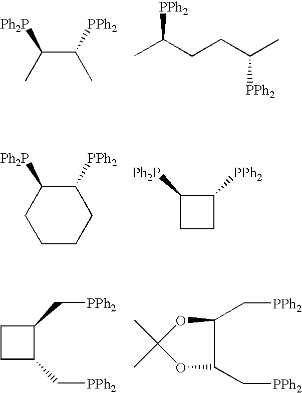

where Ph represent phenyl,
or one of the enantiomeric forms of these structures.

Substrates of another type which can be reduced are those which correspond to the following formula:

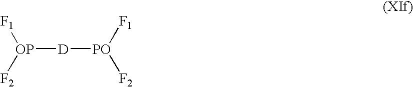

in which:
D is as defined above for A in formula (XId);
F₁ and F₂ are identical and represent a saturated aliphatic hydrocarbon-based group, said group carrying at least one chiral center; or a saturated carbocyclic group carrying at least one chiral center; or else
F₁, and F₂ together form a saturated divalent aliphatic hydrocarbon-based chain optionally interrupted with two groups X, X being as defined above for formula (XIb); two of the carbons of said chain constituting asymmetrical centers.

In formula (XIf), the various symbols represent more particularly:
D represents a C₁-C₆ alkylene chain optionally substituted with one or more (C₁-C₆)alkoxy, di(C₁-C₆)alkylamino or (C₁-C₆)alkylthio groups; a (C₃-C₈)cycloalkylene group optionally substituted with one or more (C₁-C₆)alkoxy, di(C₁-C₆)alkylamino or (C₁-C₆)alkylthio groups; a (C₆-C₁₀)arylene group optionally substituted with one or more (C₁-C₆)alkoxy, di(C₁-C₆)alkylamino or (C₁-C₆)alkythio groups; or a group —(CH₂)ⱼ—B″-(CH₂)ⱼ— where j represents an integer between 1 and 3 and B″ represents (C₃-C₈)cycloalkylene (optionally substituted with one or more (C₁-C₆)alkoxy, di(C₁-C₆)alkylamino or (C₁-C₆)alkylthio) or (C₆-C₁₀)arylene (optionally substituted with one or more (C₁-C₆)alkoxy, di(C₁-C₆)alkylamino or (C₁-C₆)alkylthio);
F₁ and F₂ are identical and represent (C₁-C₆)alkyl optionally substituted with one or more (C₁-C₆)alkoxy, said alkyl group carrying at least one chiral center; (C₃-C₈)cycloalkyl substituted with one or more (C₁-C₆)alkoxy or (C₁-C₆)alkyl, said cycloalkyl carrying at least one chiral center; or else F₁ and F₂ together form a (C₁-C₆)alkylene chain optionally interrupted with two oxygen or sulfur atoms, said chain being substituted with one or more (C₁-C₆)alkyl or (C₁-C₆)alkoxy groups, two of the carbons of said chain constituting asymmetrical centers.

An example of diphosphine oxide corresponding to formula (XIf) is:

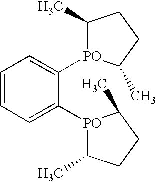

or some diastereoisomeric form.

According to another embodiment of the invention, the diphosphine oxide corresponds to one of the formulae below:

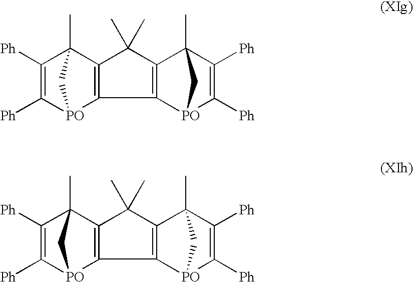

These compounds and the methods for preparing them are in particular described in application EP-A 1 064 244.

According to yet another embodiment, the diphosphine oxide has the formula:

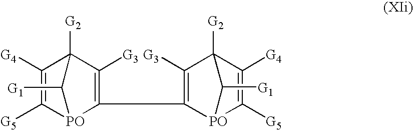

in which:
G₁, G₂, G₃, G₄ and G₅, which may be identical or different, represent a hydrogen atom or an optionally substituted hydrocarbon-based group having from 1 to 40 carbon atoms, which may be a linear or branched, saturated or unsaturated, acyclic aliphatic group; a monocyclic or polycyclic, saturated, unsaturated or aromatic, carbocyclic or heterocyclic group; or a linear or branched, saturated or unsaturated aliphatic group carrying a cyclic substituent,
G₂ and G₃ can form, together with the carbon atoms which carry them, a saturated or unsaturated ring,
G₅ can represent a group of type

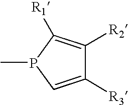

in which G₁′, G₂′ and G₃′ have the same meaning as that given for G₁, G₂ and G₃,
G₄ and G₅ cannot simultaneously represent a phenyl group.

These compounds and the methods for preparing them are described in application EP-A 0 968 220.

Examples of diphosphines corresponding to formula (XIi) above are more specifically the following compounds:

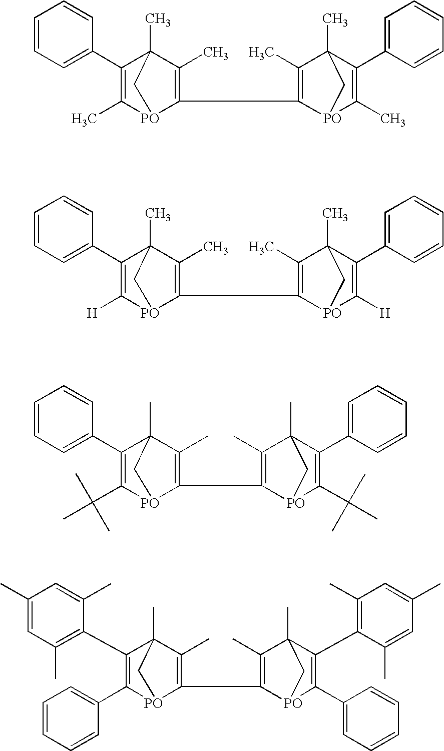

The amount of the compound of formula (I) to be used, expressed relative to the amount of substrate to be reduced, is at least equal to the stoichiometry. Thus, the ratio of the number of moles of the substrate to be reduced to the number of moles of the compound of formula (I) can range to a large extent between 1 and 1000, preferably between 1 and 50.

A Lewis acid is involved in the method of the invention.

In the present text, the term “Lewis acid” signifies a compound comprising a metal or metalloid cation that is an electron doublet acceptor, which reacts with the compound of the formula (I).

As metal or metalloid cations that are suitable for the invention, mention may particularly be made of those of the metal or metalloid elements of groups (IVa), (VIIa), (Ib), (IIb), (IIIb) and (VIII) of the Periodic Table of Elements.

In the present text, reference is hereinafter made to the Periodic Table of Elements published in the Bulletin of the Chemical Society of France, No. 1 (1966).

By way of examples of cations that are very suitable for the method of the invention, mention may more particularly be made of, among those of group (IVa), titanium, zirconium and hafnium; of group (VIIa), manganese; of group (Ib), copper; of group (IIb), zinc; of group (IIIb), boron and aluminum; of group (VIII), iron, cobalt and nickel.

Among the abovementioned cations, titanium is preferably chosen.

As more specific examples of anions, mention may in particular be made of organic anions such as carboxylates, preferably acetate, propionate, benzoate; sulfonates, preferably methanesulfonate, trifluoro-methanesulfonate; alkoxides, preferably methoxide, ethoxide, propoxide, isopropoxide; and acetyl acetonate.

As regards inorganic anions, mention may in particular be made of chloride, bromide, iodide and carbonate.

An organic anion is advantageously chosen.

There may be the presence of hydrocarbon-based groups through alkyl groups preferably having from 1 to 4 carbon atoms or cyclopentadienyl groups.

As catalysts, use is preferably made of anhydrous compounds.

Examples of compounds which can be used in the method of the invention are given hereinafter:
iron compound:
   iron (II) acetate
   iron (II) acetyl acetonate
   iron (II) bromide
   iron (III) bromide
   iron (III) chloride
   iron (III) ethoxide
   iron (III) i-propoxide
   iron (III) stearate
   iron (III) trifluoroacetyl acetonate
cobalt compound:
   cobalt (II) acetate
   cobalt (II) acetyl acetonate
   bis(cyclopentadienyl)cobalt (II)
   cobalt (II) bromide
   cobalt (II) chloride
   cobalt (II) citrate
   cobalt (II) cyclohexanebutyrate
   cobalt (II) 2-ethylhexanoate
   (1R,2R)-(−)-1,2-cyclohexanediamino-N,N′-bis(3,5-di-t-butylsalicylidene) cobalt (II)
nickel compound:
   nickel (II) acetyl acetonate
   nickel (II) bromide
   nickel (II) chloride
   nickel (II) hexafluoroacetyl acetonate
titanium compound:
   bis(cyclopentadienyl)titanium dichloride
   bis(ethylcyclopentadienyl)titanium dichloride
   titanium chloride triisopropoxide
   cyclopentadienyltitanium trichloride
   pentamethylcyclopentadienyltitanium trimethoxide
   titanium (IV) bromide
   titanium (IV) n-butoxide
   titanium (IV) t-butoxide
   titanium (IV) chloride
   titanium (di-i-propoxide)bis(acetyl acetonate)
   titanium ethoxide
   titanium (III) fluoride
   titanium (IV) i-propoxide
zirconium compound:
   bis(cyclopentadienyl)zirconium dichloride
   bis(tetramethylcyclopentadienyl)zirconium dichloride
   n-butyl(cyclopentadienyl)zirconium dichloride
   cyclopentadienylzirconium trichloride
   zirconium (IV) acetyl acetonate
   zirconium (IV) n-butoxide
   zirconium (IV) t-butoxide
   zirconium (IV) n-propoxide
   zirconium (IV) trifluoroacetyl acetonate
hafnium compound:
   hafnium (IV) acetyl acetonate
   hafnium (IV) bromide
   hafnium (IV) t-butoxide
   hafnium (IV) chloride
   hafnium (IV) ethoxide
   hafnium (IV) propoxide monoisopropoxide
boron compound:
   boron bromide
   tri-n-amylborate
   tri-n-butylborate
copper compound:
   copper (II) acetate
   copper (II) acetyl acetonate
   copper (II) bromide
   copper (II) ethyl acetoacetate
   copper (II) ethylhexanoate
   copper (II) fluoride
   copper (II) formate
   copper (II) oxalate
   copper (II) tartrate
   copper (II) trifluoroacetyl acetonate
   copper (II) trifluoromethanesulfonate
aluminum compound:
   aluminum ethoxide
   aluminum fluoride
   aluminum hexafluoroacetyl acetonate
   aluminum i-propoxide
zinc compound:
   zinc acetate
   zinc acetyl acetonate
   zinc bromide
   zinc chloride
   zinc cyclohexanebutyrate
   zinc 2-ethylhexanoate
   zinc fluoride
   zinc iodide
   zinc trifluoromethanesulfonate
manganese compound:
   bis(cyclopentadienyl)manganese
   bis(ethylcyclopentadienyl)manganese
   bis(pentamethylcyclopentadienyl)manganese
   bis(i-propylcyclopentadienyl)manganese
   bis(tetramethylcyclopentadienyl)manganese
   (1R,2R)-(−)-[1,2-cyclohexanediamino-N,N′
   bis(3,5-di-t-butylsalicylidene)]manganese (III) chloride
   (1S,2S)-(+)-[1,2-cyclohexanediamino-N,N′-bis(3,5-di-t-butylsalicylidene)]manganese (III) chloride
   tricarbonylcyclopentadienylmanganese
   manganese (III) acetyl acetonate
   manganese (II) bromide
   manganese (II) carbonate
   manganese (II) chloride
   manganese (II) cyclohexanebutyrate
   manganese (II) 2-ethylhexanoate
   manganese (II) fluoride
   manganese (II) iodide
   pentacarbonylmanganese bromide
   manganese (III) phthalocyanin
   tricarbonylmethylcyclopentadienylmanganese.

As more specific examples of Lewis acids, mention may be made of titanium isopropoxide or zinc trifluoroacetate.

The amount of catalyst used, expressed by the ratio of the number of moles of Lewis acid to the number of moles of substrate to be reduced, ranges between 0.1 and 1, and is preferably in the region of 0.5.

The method of the invention is preferably carried out in an organic solvent.

It is also possible for one of the excess reactants to serve as reaction solvent.

A solvent which is inert under the reaction conditions and which preferably solubilizes the reactants is used.

Advantageously, the solvent is chosen such that it has a high boiling point (preferably above 80° C.).

As examples of organic solvents that are suitable for the invention, mention may in particular be made of halogenated or nonhalogenated, aliphatic, cycloaliphatic or aromatic hydrocarbons; ethers and alcohols.

By way of nonlimiting examples of solvents, mention may be made of:
aliphatic and cycloaliphatic hydrocarbons, more particularly paraffins, such as, in particular, hexane, heptane, octane, isooctane, nonane, decane, undecane, tetradecane, petroleum ether and cyclohexane; aromatic hydrocarbons such as, in particular, benzene, toluene, xylenes, ethylbenzene, diethylbenzenes, trimethylbenzenes, cumene, pseudocumene, petroleum cuts consisting of a mixture of alkylbenzenes, in particular cuts of the Solvesso® type,
aliphatic or aromatic halogenated hydrocarbons, and mention may be made of: perchlorinated hydrocarbons such as, in particular, trichloromethane, tetrachloroethylene; partially chlorinated hydrocarbons such as dichloromethane, dichloroethane, tetra-chloroethane, trichloroethylene, 1-chloro-butane, 1,2-dichlorobutane; monochlorobenzene, 1,2-dichlorobenzene, 1,3-dichlorobenzene, 1,4-dichlorobenzene, or mixtures of various chlorobenzenes,
aliphatic, cycloaliphatic or aromatic ether oxides, and more particularly methyl tert-butyl ether, dipentyl oxide, diisopentyl oxide, ethylene glycol dimethyl ether (or 1,2-dimethoxyethane), diethylene glycol dimethyl ether (or 1,5-dimethoxy-3-oxapentane), or cyclic ethers, for example dioxane, tetrahydrofuran,
aliphatic or cycloaliphatic alcohols, more particularly ethanol, isopropanol, butanol, isobutanol, hexanol, cyclohexanol or ethylene glycol.

Toluene is advantageously chosen.

The amount of organic solvent used is such that the concentration of the substrate is advantageously between 0.1 and 1 mol/liter, preferably in the range of 0.5 mol/liter.

In accordance with the method of the invention, the substrate to be reduced is brought into contact with the compound of formula (I), in the presence of the catalyst and preferably in an organic solvent.

As regards the temperature and pressure conditions, they are advantageously as described below.

The reduction reaction is generally carried out at a temperature ranging between ambient temperature and 150° C., preferably between 80 and 120° C.

The term “ambient temperature” is intended to mean a temperature most commonly between 15° C. and 25° C.

The reduction time can vary to a large extent between 2 hours and 24 hours, depending on the amount of catalyst used and the reaction temperature.

The method of the invention is carried out under atmospheric pressure, but preferably under a controlled atmosphere of inert gases such as nitrogen or rare gases, for example argon. A pressure slightly above or below atmospheric pressure may be suitable.

As regards the methods of practical execution of the invention, a preferred method consists in loading the substrate to be reduced, the organic solvent and the Lewis acid-type catalyst, and then introducing the reducing compound of formula (I).

At the end of the reaction, the reduced product is recovered.

Conventional means can be used for this purpose.

If the reduced product is in liquid form, the reaction medium can, for example, be treated, at the end of the reaction, with a basic solution in order to hydrolyze the hydrides that have not reacted.

The base is preferably an alkali metal hydroxide, and more preferably sodium hydroxide or potassium hydroxide.

A basic solution having a concentration ranging from 1 to 5 N is advantageously used.

The amount of base used is at least equal to the stoichiometric amount expressed relative to the reduced product obtained, and can be in an excess which can reach 100% of the stoichiometric amount.

After the treatment with a base, the aqueous and organic phases are separated.

The organic phase, which comprises the excess compound (I) (which is then hydrolyzed) which has not reacted and the reduced product obtained, is recovered. The organic solvent is evaporated off.

The reduced product is obtained, for example, by distillation.

When the product is solid, a treatment with a base as described above is carried out.

The aqueous and organic phases are separated.

The organic phase is recovered and the organic solvent is evaporated off.

The solid is washed with an organic solvent in order to remove the traces of solvent, for example an aliphatic hydrocarbon, such as, in particular, pentane.

The product is recovered and is then dried. The drying temperature depends on the melting point of the product obtained. The drying is generally carried out in the air at a temperature ranging between ambient temperature and 100° C.

Thus, in accordance with the method of the invention, the reduction of the compounds of formula (I) to (XII) results, respectively, in the following compounds (I′) to (XII′):

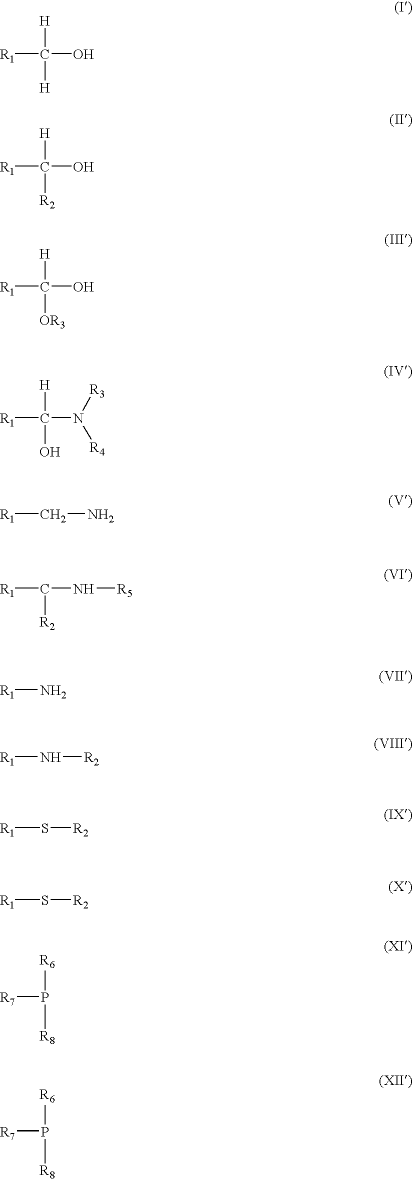

An example of realization of the invention, given by way of illustration and which is in no way limiting in nature, is given hereinafter.

In the examples, the yield (RR) is defined as the ratio of the number of moles of product formed to the number of moles of substrate involved.

### EXAMPLE 1

### 1. Preparation of BINAPO

It is prepared by oxidation of BINAP.

(S)— or (R)-BINAP (2,2′-bis(diphenylphosphino)-1,1′-binaphthyl) (3 g, 4.81 mmol, 1 eq.) dissolved in 100 ml of CH₂Cl₂ is placed in a 250 ml round-bottomed flask.

It is cooled to 0° C. and 10 ml of aqueous hydrogen peroxide at 35% by weight are added.

The mixture is stirred, while allowing the temperature to return to ambient temperature, for 4 hours.

100 ml of water are then added.

The organic phase is separated and the aqueous phase is extracted with CH₂Cl₂.

The combined organic phases are washed with saturated sodium bisulfite.

The absence of peroxide is verified and then drying over sodium sulfate and evaporation are performed.

A white solid is obtained (m=3.14 g, 4.8 mmol, quantitative yield).

The characterization of the diphosphine in the form of dioxide (BINAPO) is as follows:

¹H NMR (300 MHz, CDCl₃): 6.80 (d, 4H, J=3.7), 7.2-7.3 (m, 8H), 7.3-7.5 (m, 12H) 7.6-7.7 (m, 4H), 7.8-7.9 (m, 4H).

³¹P NMR (81 MHz, CDCl₃): 28.67

Mp: 256-258° C.

### 2. Reduction of BINAPO

The BINAP oxide (300 mg, 0.46 mmol, 1 eq.) is placed in a reaction tube equipped with a stirrer and under an inert atmosphere.

2 ml of toluene and (0.5 ml, 2.8 mmol, 6 eq.) of tetra-methyldisiloxane and (0.065 ml, 0.23 mmol, 0.5 eq.) of titanium isopropoxide are then added.

The reaction mixture is then heated at 85° C. and stirred for 20 hours.

It is cooled and 1 ml of sodium hydroxide (3N) is added.

The mixture is allowed to stir for 15 minutes and then 5 ml of dichloromethane are added.

The mixture is filtered.

The organic phase is recovered and then dried and evaporated so as to obtain 280 mg of a white solid.

The solid is taken up in 3 ml of pentane and filtered over a sintered glass funnel.

A white solid of BINAP is obtained.

260 mg of product are recovered, which corresponds to a yield of 91%.

The product obtained, BINAP, has the following NMR characteristics:

m=260 mg.

¹H NMR (300 MHz, CDCl₃): 6.80 (d, 4H, J=3.7), 7.2-7.3 (m, 8H), 7.3-7.5 (m, 12H), 7.6-7.7 (m, 4H), 7.8-7.9 (m, 4H).

³¹P NMR (81 MHz, CDCl₃): −14.63.

### EXAMPLE 2

### 1. Preparation of 4,4′-dicyanoBINAPO

It is prepared by bromination, in the 4,4′-position, of BINAPO, and then nucleophilic substitution of the bromine atoms with cyano groups.

### Preparation of 4,4′-dibromoBINAPO

BINAPO (5 g, 7.64 mmol, 1 eq.) dissolved in 150 ml of dichloromethane is placed in a dry 250 ml round-bottomed flask.

Pyridine (0.62 ml, 7.64 mmol, 1 eq.) is then added, followed by dibromine (1.2 ml, 22.92 mmol, 3 eq.).

The mixture is stirred at ambient temperature for 20 hours.

The mixture is transferred into a separating funnel and is then successively treated with saturated sodium bisulfite, brine, and then saturated sodium bicarbonate.

Drying over sodium sulfate and evaporation are performed. The procedure is repeated twice.

The product is recrystallized from methanol so as to obtain a white solid (m=4.74 g, 5.8 mmol, i.e. a yield of 76%).

The characterization of the diphosphine in dibrominated form is as follows:

¹H NMR (300 MHz, CDCl₃): 6.80 (d, 2H, J=8.3; 8.8′H), 6.85 (ddd, 2H, J=0.9; 6.7; 15.1; 7.7′H), 7.2-7.5 (m, 18H, phenyl+H6 and H6′), 7.6-7.7 (m, 4H; phenyl), 7.75 (s, 2H, 3.3′H), 8.23 (d, 2H, J=8.4; 5.5′H).

³¹P NMR (81 MHz, CDCl₃): 27.60

Mp: >300° C.

### Preparation of 4,4′-dicyanoBINAPO

4,4′-dibromoBINAPO (200 mg, 0.25 mmol, 1 eq.) and copper cyanide (63 mg, 0.7 mmol, 2.8 eq.) are placed, under an inert atmosphere, in a 50 ml round-bottomed flask equipped with a condenser.

The mixture is dissolved in 3 ml of DMF and refluxed overnight.

The mixture is cooled and then treated with a solution of ethylenediamine (1 ml) and water (1 ml).

The mixture is stirred for 2 minutes and then 5 ml of water and 10 ml of toluene are added.

The mixture is stirred for 5 minutes and then the aqueous phase is extracted once with toluene.

The combined organic phases are washed successively once with water, 4 times with HCl (0.1 M), once with brine, and then once with saturated sodium bicarbonate.

The product is then dried over sodium sulfate and then evaporated under reduced pressure (approximately 8 mm of mercury).

The solid obtained is recrystallized from methanol.

A white solid which is pure is obtained (m=0.100 g, 0.15 mmol, i.e. a yield of 60%).

The characterization of the diphosphine (PO) in dicyanated form is as follows:

¹H NMR (200 MHz, CDCl₃): 6.73 (d, 2H, J=8.4), 6.90 (ddd, 2H, J=1.0; 7.0; 14.3), 7.2-7.8 (m, 22H), 7.85 (d, 2H, J=11.3), 8.28 (d, 2H, 8.3).

³¹P NMR (81 MHz, CDCl₃): 27.77

Mass (ESI⁺) MH⁺=813.35

Mp: >300° C.

### 2. Reduction of 4,4′-dicyanoBINAPO

The 4,4′-dicyanoBINAPO (300 mg, 0.44 mmol, 1 eq.) is placed in a reaction tube equipped with a stirrer and under an inert atmosphere.

2 ml of toluene and (0.5 ml, 2.8 mmol, 6 eq.) of tetra-methyldisiloxane and (0.13 ml, 0.46 mmol, 1 eq.) of titanium isopropoxide are then added.

The reaction mixture is then heated at 110° C. and stirred for 20 hours.

It is cooled and 1 ml of sodium hydroxide (3N) is added.

The mixture is left to stir for 2 hours and then 5 ml of dichloromethane are added. The mixture is filtered.

The organic phase is recovered and then dried and evaporated so as to obtain 289 mg of a white solid.

The solid is taken up in 3 ml of pentane and filtered over a sintered glass funnel.

A white solid of 4,4′-diamBINAP is obtained.

272 mg of product are recovered, which corresponds to a yield of 91%.

The product obtained, 4,4′-diamBINAP, has the following NMR characteristics:

¹H NMR (300 MHz, CDCl₃): 6.64 (d, 2H, J=9), 6.93-6.97 (m, 2H), 7.1-7.3 (m, 20H), 7.54 (t, 2H), 7.98 (s, 2H), 8.23 (d, 2H, J=8.3).

³¹P NMR (81 MHz, CDCl₃): −13.30.

### EXAMPLE 3

### Reduction of Benzonitrile

The benzonitrile (0.103 ml, 1 mmol, 1 eq.) is placed in a reaction tube equipped with a stirrer and under an inert atmosphere.

2 ml of toluene and (1.8 ml, 10 mmol, 10 eq.) of tetra-methyldisiloxane and (0.30 ml, 1 mmol, 1 eq.) of titanium isopropoxide are then added.

The reaction mixture is then heated at 120° C. and stirred for 30 hours.

It is cooled and 1 ml of sodium hydroxide (3N) is added.

The mixture is left to stir for 5 hours and 5 ml of dichloromethane are then added.

The mixture is filtered. The organic phase is recovered and then dried and evaporated. The residue obtained is distilled so as to obtain a transparent liquid (64 mg, 60%) corresponding to benzylamine.

The product obtained, 4,4′-diamBINAP, has the following

NMR characteristics:

¹H NMR (300 MHz, CDCl₃): 7.2-7.45 (m, 5H), 3.85 (s, 2H), 1.77 (s, 2H).

1-21. (canceled)

22. A method of reducing an oxidized functional group, present in a substrate, to a lower oxidation state, comprising the steps of exposing the substrate to a siloxane compound corresponding to the following formula (I), combined with an effective amount of a Lewis acid-type catalyst 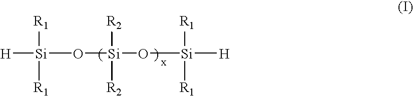
wherein:
R₁ and R₂, which are identical or different, represent an alkyl, cycloalkyl or aryl group, and
x is a number ranging from 0 to 50,
said oxidized group being carboxylic acid, ester, amide, nitrile, imine, nitro, nitrogen oxide, sulfoxide, sulfone, phosphine oxide or phosphine sulfide.

23. The method as claimed in claim 22, wherein in formula (I), R₁ and R₂ are identical.

24. The method as claimed in claim 22, wherein the siloxane compound corresponds to formula (I) in which R₁ and R₂ represent an alkyl group having from 1 to 4 carbon atoms.

25. The method as claimed in claim 22, wherein the siloxane compound corresponds to formula (I) in which x is between 0 and 10, optionally equal to 0 or 1.

26. The method as claimed in claim 22, wherein the siloxane compound is tetramethyldisiloxane.

27. The method as claimed in claim 22, wherein said groups is carried by an aliphatic chain or a ring, or is included in a ring.

28. The method as claimed in claim 27, wherein the substrate comprising the function to be reduced is represented by one of the formulae: 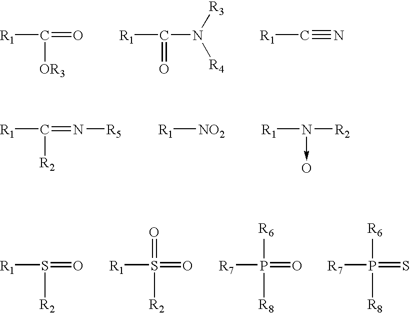
wherein,
R₁ to R₈ represent a hydrocarbon-based group having from 1 to 20 carbon atoms,
R₃, R₄ and R₅ also represent a hydrogen atom,
at most one of the groups R₆, R₇ and R₈ represent a hydrogen atom, and, optionally R₁ and R₂, R₁ and R₅, R₆ and R₇, R₇ and R₈, and R₆ and R₈ are linked together so as to form a ring.

29. The method as claimed in claim 28, wherein the substrate to be reduced is benzonitrile, a phosphine oxide, a diphosphine in the form of a dioxide, optionally BINAPO, or an oxide derived from BINAP substituted in the 6- and 6′-position, 5- and 5′-position or 4- and 4′-position, optionally with nitrile groups.

30. The method as claimed in claim 22, wherein the number of moles of the substrate to be reduced to the number of moles of the compound of formula (I) presents a ratio ranging between 1 and 1000, optionally between 1 and 50.

31. The method as claimed in claim 22, wherein the catalyst is a compound comprising a metal or metalloid cation of the metal or metalloid elements of groups (IVa), (VIIa), (Ib), (IIb), (IIIb) and (VIII) of the Periodic Table of Elements.

32. The method as claimed in claim 31, wherein the metal or metalloid element is titanium, zirconium and hafnium; manganese; copper; zinc; boron and aluminum; iron, cobalt or nickel.

33. The method as claimed in claim 31, wherein the anion is carboxylate, optionally acetate, propionate, benzoate; sulfonate, optionally methanesulfonate, trifluoromethanesulfonate; alkoxide, optionally methoxide, ethoxide, propoxide, isopropoxide; or acetyl acetonate.

34. The method as claimed in claim 31, wherein the anion is chloride, bromide, iodide or carbonate.

35. The method as claimed in claim 31, wherein the catalyst is titanium isopropoxide or zinc trifluoroacetate.

36. The method as claimed in claim 31, wherein the catalyst used, expressed by the ratio of the number of moles of Lewis acid to the number of moles of substrate to be reduced, is used in an amount of ranging between 0.1 and 1, optionally in the region of 0.5.

37. The method as claimed in claim 22, wherein the reaction is carried out in an organic solvent, optionally a halogenated or nonhalogenated, aliphatic, cycloaliphatic or aromatic hydrocarbon; an ether or an alcohol.

38. The method as claimed in claim 37, wherein the solvent is toluene.

39. The method as claimed in claim 22, wherein the reduction reaction is carried out at a temperature ranging between ambient temperature and 150° C., optionally between 80 and 120° C.

40. The method as claimed in claim 22, carried out under atmospheric pressure, optionally under a controlled atmosphere of inert gases, optionally nitrogen.

41. The method as claimed in one claim 22, wherein the substrate to be reduced, the organic solvent and the Lewis acid-type catalyst are loaded and then the reducing compound of formula (I) is added.

42. The method as claimed in claim 22, wherein a basic hydrolysis is carried out at the end of the reaction and the reduced product is recovered.
